(19) United States
(12) Patent Application Publication
(10) Pub. No.: US 2010/0076226 A1
(43) Pub. Date: Mar. 25, 2010

(54) Preparation of Dibutoxymethane

(76) Inventors: Todd Coleman, Batesville, AR (US); Allen Blankenship, Batesville, AR (US); Eddie Eckart, Batesville, AR (US)

Correspondence Address: LEWIS, RICE & FINGERSH, LC;ATTN: BOX IP DEPT., 500 NORTH BROADWAY, SUITE 2000, ST LOUIS, MO, 63102, US

(21) Appl. No.: 12/544,882

(22) Filed: Aug. 20, 2009

Related U.S. Application Data
(60) Provisional application No. 61/090,275, filed on Aug. 20, 2008.

(51) Int. Cl. C07C 43/30; C07C 41/01

(52) U.S. Cl. 568/594

(57) ABSTRACT

A process for the preparation of dibutoxymethane, in one embodiment from 50% formaldehyde, in a condensation reaction without the use of co-solvent.

### CROSS REFERENCE TO RELATED APPLICATION(S)

This application claims benefit of U.S. Provisional Patent Application Ser. No. 61/090,275 filed Aug. 20, 2008, the entire disclosure of which is herein incorporated by reference.

### BACKGROUND

1. Field of the Invention

This disclosure relates to the field of processes for the creation of dibutoxymethane (“DBM”). Specifically, to the field of processes of creating DBM from 50% formaldehyde.

2. Description of the Related Art

DBM, also known as n-Butylal, is a commonly known product to those skilled in the art. DBM has been found to be useful to reduce particulate emissions from diesel fuel combustion and improve the cetane value of diesel fuel (WO 86/03511). DBM has also been found to be a good solvent for foundry core aggregate and binders (U.S. Pat. No. 4,051,092).

Other analogous compounds to DBM are also known. For example, Dimethoxymethane (“DMM”) is an item of commerce used extensively in the cosmetic industry. Processes and methods for the preparation and purification of DMM are found in U.S. Pat. Nos. 4,385,965; 5,051,153; 6,015,875; 6,160,185; 6,379,507 and Swiss Patent CH 688 041.

Diethexymethane (“DEM”) is another commonly known analogous product to DBM to those skilled in the art. Like, DMM, DEM is an item of commerce. Its main use is as a solvent. Processes and methods for its use and purification are also well established and are found in U.S. Pat. Nos. 4,613,411 and 4,740,273.

While DMM, DEM, and DBM are similar in structure, they have vastly different properties. The boiling points of the materials increase dramatically with increasing molecular weight. In addition, their solubility in water decreases with increasing molecular weight. These compounds also form azeotropes with water and the corresponding alcohol at different compositions and boiling points. Thus, DMM, DEM, and DBM all have different boiling points, solubility properties, and azeotrope qualities. Therefore, one process cannot be used to prepare all three analogs.

As opposed to the processes and methods of DMM and DEM, very little information is known or found in the public literature on the preparation of DBM. WO 86/03511 describes a process that uses butanol, aqueous formaldehyde, a cationic exchange resin, and benzene. Benzene is used to remove the water from the formaldehyde and the water formed in the reaction azeotropic distillation. Other solvents such as toluene, hexane, or heptane could also be used to accomplish the purpose. The significant disadvantage of this process however is that the inert distilling agent must be removed from the desired product. That is, the process requires a co-solvent for distillation purposes which must be removed from the end product. This greatly increases the costs of the procedure (via the added cost of the co-solvent and cost associated with co-solvent removal) and the complexity of the manufacturing process.

### SUMMARY

The following is a summary of the invention in order to provide a basic understanding of some aspects of the invention. This summary is not intended to identify key or critical elements of the invention or to delineate the scope of the invention. The sole purpose of this section is to present some concepts of the invention in a simplified form as a prelude to the more detailed description that is presented later.

Because of these and other problems in the art disclosed herein, among other things, is a new process for the preparation dibutoxymethane, in one embodiment from 50% formaldehyde.

In one embodiment, the method comprises the steps of: (1) reacting paraformaldehyde and butanol in a condensation reaction without the use of a co-solvent for the removal of water. This method for the preparation of dibutoxymethane can also be performed with a recycled butanol discharge.

Also disclosed herein is dibutoxymethane formed by the process of: (1) providing a paraformaldehyde and butanol; (2) reacting said paraformaldehyde and said butanol in a condensation reaction without the use of a co-solvent for the removal of water; and (3) segregating said dibutoxymethane. Again, this method for the preparation of dibutoxymethane can be performed with a recycled butanol discharge.

Also disclosed herein is a method for the production of dibutoxymethane, without a recycled butanol discharge, the method comprising the steps of: (1) charging water, methanol, paraformaldehyde, and a condensation reaction catalyst together to create a mixture; (2) heating said mixture to a temperature at which a clear solution is obtained; (3) charging virgin butanol to said mixture; (4) heating said mixture; (5) collecting water and butanol distillate in a distillation trap; (6) cooling said mixture when the accumulation of said water distillate ceases; (7) charging a neutralization agent to said mixture; (8) charging water to said mixture; (8) re-heating the mixture to initiate distillation; (10) continuing distillation until water has been collected in a receiver; (11) separating a top and a bottom phase of a distillate; and (12) cooling a mixture residue.

In one embodiment of this method, 60-ml of deionized water is charged in the step of charging.

In another embodiment of this method, 1.2 grams of methanol is charged in the step of charging.

In another embodiment of this method, 60 grams of paraformaldehyde is charged in the step of charging.

In another embodiment of this method, 0.1 mls of 98% sulfuric acid is charged as the condensation reaction catalyst in the step of charging.

In another embodiment of this method, the mixture is heated to a temperature of 90-100° C. in the step of heating the mixture to a temperature at which a clear solution is obtained.

In yet another embodiment of this method, 370 grams of virgin butanol is charged to the mixture in the step of charging.

In another embodiment of this method, the mixture is heated to a temperature of 125° C. in the step of heating the mixture.

In another embodiment of this method, the mixture is cooled to 50° C. in the step of cooling the mixture when the accumulation of the water distillate ceases.

In another embodiment of this method, 0.1-ml of PM 16 is charged to the mixture in the step of charging a neutralization agent to the mixture.

In yet another embodiment of this method, 110 grams of water is charged to the mixture in the step of charging water to the mixture.

In another embodiment of this method, the mixture is re-heated to about 125° C. in the step of re-heating the mixture to initiate distillation.

In another embodiment of this method, wherein the distillation of the mixture continues until about 100 ml of water has been collected in the step of continuing distillation until water has been collected in a receiver.

In still yet another embodiment of this method, the mixture residue is cooled to less than 50° C. in the step of cooling a mixture residue.

Another method for the production of dibutoxymethane disclosed herein uses a recycled butanol discharge, and comprises the steps of: (1) charging water, methanol, paraformaldehyde, and a condensation reaction catalyst together to create a mixture; (2) heating the mixture to a temperature at which a clear solution is obtained; (3) charging the top layer of the distillate trap from the previous batch and calculating the amount of butanol therein; (4) charging the top layer from the receiver distillate from the previous batch and calculating the amount of butanol therein; (5) calculating the amount of butanol to be charged; (6) charging the amount of virgin butanol calculated in the step of charging to the mixture; (7) heating the mixture; (8) collecting water and butanol distillate in a distillation trap; (9) cooling the mixture when the accumulation of the water distillate ceases; (10) charging a neutralization agent to the mixture; (11) charging water to the mixture; (12) re-heating the mixture to initiate distillation; (13) continuing distillation until water has been collected in a receiver; (14) separating a top and a bottom phase of a distillate; and (15) cooling a mixture residue.

### BRIEF DESCRIPTION OF THE DRAWINGS

FIG. 1 provides an embodiment of a flowchart of a process for the preparation of dibutoxymethane and provides molecular diagrams of the molecules.

FIG. 2 provides an embodiment of a flow chart of a process for the preparation of dibutoxymethane from formaldehyde without the use of a co-solvent.

FIG. 3 provides an embodiment of a flow chart of an exemplary step-by-step bench process for the preparation of dibutoxymethane with virgin butanol.

FIG. 4 provides another embodiment of a flow chart of an exemplary step-by-step bench process for the preparation of dibutoxymethane with a distillate residue recycle.

FIG. 5 provides an embodiment of a chart of the raw materials used in the preparation of dibutoxymethane, in the process of FIG. 1.

FIG. 6 provides an embodiment of a process flow diagram for the continuous production of dibutoxymethane.

FIG. 7 provides an embodiment of a process flow diagram for the batch production of dibutoxymethane.

### DESCRIPTION OF PREFERRED EMBODIMENT(S)

The following detailed description illustrates by way of example and not by way of limitation. Described herein, among other things, is a new process for the preparation dibutoxymethane in a condensation reaction, without the use of a co-solvent, in one embodiment from 30-50% formaldehyde.

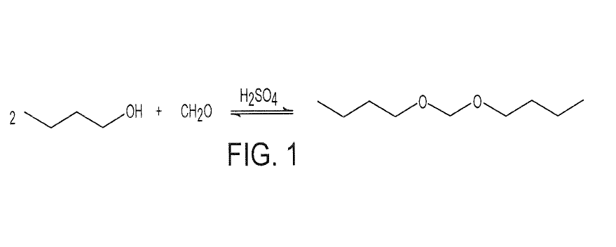

FIG. 1 shows the molecular diagram of an embodiment of a chemical process for the creation of prep crude dibutoxymethane (“DBM”) from a condensation reaction using 30-50% formaldehyde. It is contemplated in this disclosure that this process can be comprised of an embodiment in which simply virgin butanol is utilized and an embodiment in which a recycled butanol distillate is utilized. Further, it is also contemplated that the production of dibutoxymethane may be by a batch or a continuous production.

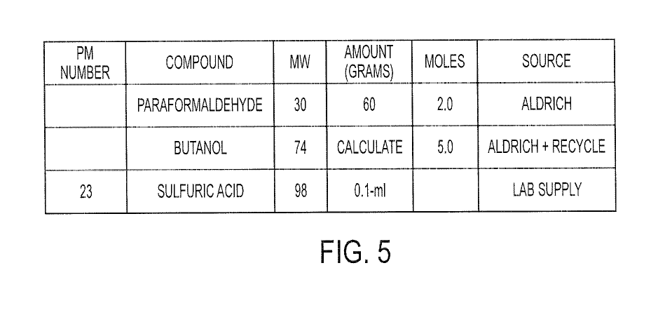

FIG. 5 shows a table of the raw materials used in one embodiment of the process for the creation of prep crude DBM using 30-50% formaldehyde. It is important to note that is contemplated that any comparable, analogous strong acid or strongly acidic ion-exchange resin known to those of skill in the art now or in the future may be used in place of the sulfuric acid identified in the table. Identification of this particular chemical is in no way determinative. Further, the disclosed MW, amounts, and moles are not determinative, and any MW, amounts or moles known to those of skill in the art that would effectively function in the disclosed process are contemplated in the process of this disclosure.

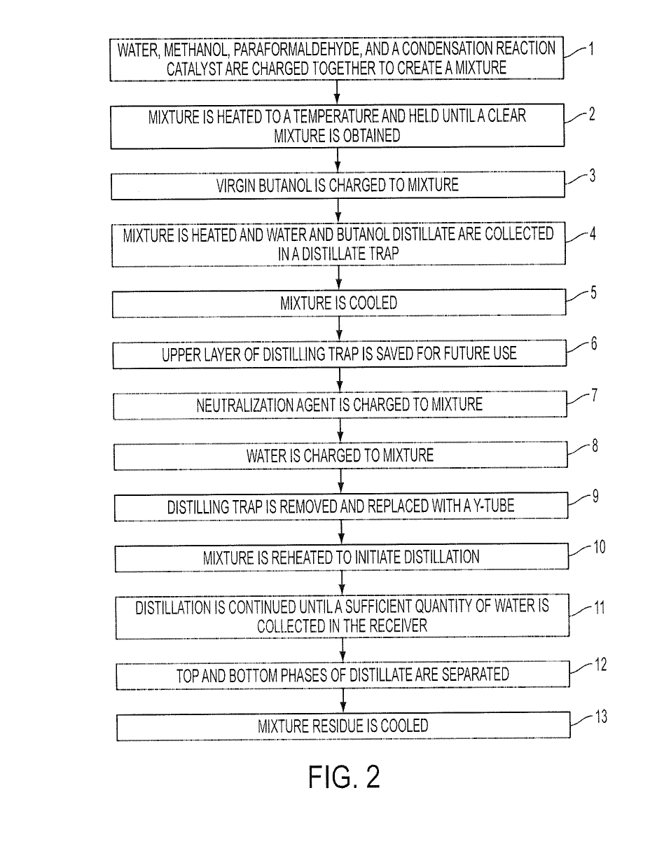

An embodiment of the disclosed process for the preparation of dibutoxymethane in a condensation reaction, without the use of a co-solvent, from 30-50% formaldehyde is provided in the flow-chart of FIG. 2. As a preliminary matter, it is noted that at any point in this process a sample of the mixture may be taken and submitted for testing or procedures known to those of skill in the art to have utility in such a reaction. Examples of such tests and/or procedures include, but are not limited to, gas-liquid chromatography analysis.

In a first step (1) of this embodiment of the disclosed process, a 30-50% formaldehyde solution and a condensation reaction catalyst known to those of skill in the art are charged together to create a mixture. In one embodiment of the disclosed process, the condensation reaction catalyst utilized is sulfuric acid. Further, in one embodiment of the disclosed process, the 30-50% formaldehyde solution is comprised of a mixture of paraformaldehyde, methanol and water.

Then, in step (2), the mixture is heated to a temperature and held until a clear mixture is obtained.

After the clear mixture is obtained, in step (3), virgin butanol is charged to the mixture.

Next, in step (4), the mixture will be heated and the water and butanol distillate will be collected in a distilling trap known to those of skill in the art. In one embodiment of the process of FIG. 2, the distilling trap utilized will be a Dean-Stark trap.

When the accumulation of water ceases, in step (5), the mixture will be cooled.

In an embodiment of the disclosed process, after cooling, the upper layer from the distilling trap (e.g., butanol) will be saved for further use in step (6). Further, in an embodiment of the disclosed process, a sample of this upper layer may be taken and submitted for compound analysis. Generally, any method of compound analysis (e.g., gas-liquid chromatography) known to those of skill in the art is contemplated in this step of the disclosed process.

Then, in step (7), a neutralization agent known to those of skill in the art will be charged to the mixture. In one embodiment of the process of FIG. 2, the utilized neutralizing agent is 50% caustic.

Following the charging of the neutralization agent, in step (8) water will be charged to the flask.

In an embodiment of the disclosed process, after step (8), the distilling trap will be removed from the reaction and replaced with a y-tube in step (9). Alternatively, the distilling trap can be altered to allow for the removal of both butanol and water from the reaction mixture. It should be understood that any alteration that allows for the butanol and water to be removed from the mixture is contemplated.

In step (10), the mixture will be reheated to a temperature to initiate distillation.

After the initiation of distillation, in step (11), distillation is continued until a sufficient quantity of water has been collected in the receiver.

Then, in step (12), the top and bottom phases of the distillate will be separated. In one embodiment of this step, the weight and volume of each phase will be recorded and a sample will be submitted for compound analysis. While any type of compound analysis know to those of skill in the art is contemplated, in one embodiment gas-liquid chromatography will be utilized. Further, in another embodiment of the process of FIG. 2, the organic phase of the distillate will be recycled to subsequent batches.

Finally, in step (13), the mixture residue will be cooled. In one embodiment of the process of FIG. 2, after the residue is cooled, it will be transferred to a storage bottle. Further, it is contemplated in one embodiment, that a sample of this final cooled mixture residue will be taken and submitted for compound analysis. Again, while any type of compound analysis known to those of skill in the art is contemplated, in one embodiment gas-liquid chromatography will be utilized.

As noted previously, the process depicted in FIG. 2 can also be performed in an embodiment with a butanol discharge recycle. In this embodiment, the following steps are added to the process. After a clear mixture is obtained in step (2), in the embodiment of the process of FIG. 2 in which a butanol discharge recycle is utilized, the following steps are performed in place of step (3). First, the top, organic layer of the distillate from the distillate trap of the previous batch will be charged and the amount of butanol therein will be calculated. Generally, the amount of butanol in the charge will be calculated by compound analysis, such as gas-liquid chromatography. Then, the top, organic layer of the distillate from the receiver of the previous batch will be charged and the amount of butanol therein will be calculated. Again, the amount of butanol in the charge will be calculated by compound analysis, such as gas-liquid chromatography. Next, the amount of virgin butanol to be charged to the mixture is calculated by a method known to those of skill in the art. In one embodiment, this amount will be calculated by subtracting the combined net butanol charges from the distillate trap and the receiver previously calculated and subtracting this combined net butanol charge from 370. The final additional step in this embodiment of the process of FIG. 2 is charging the calculated amount of virgin butanol to the mixture.

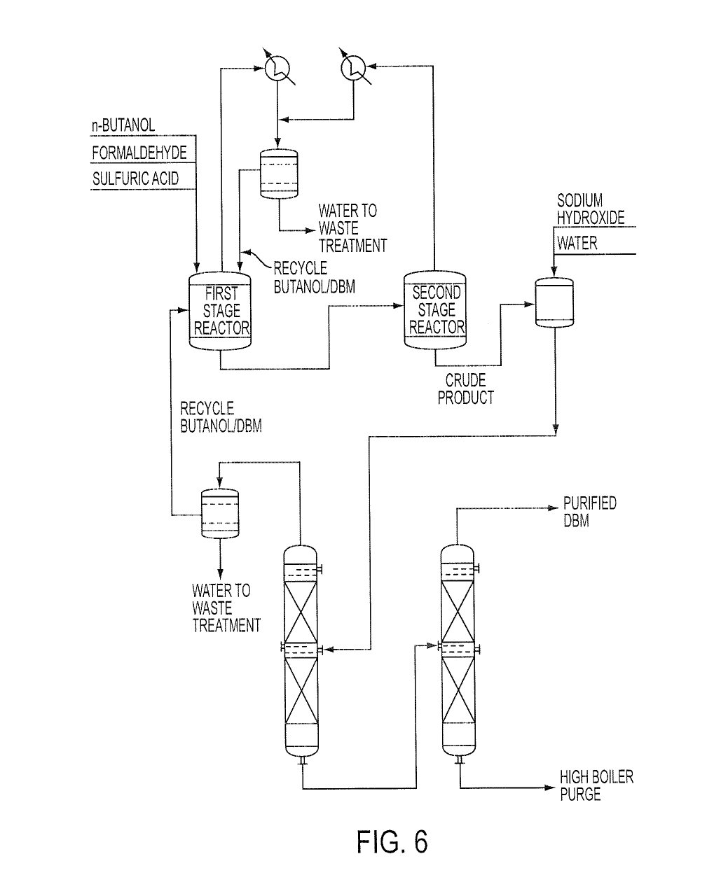

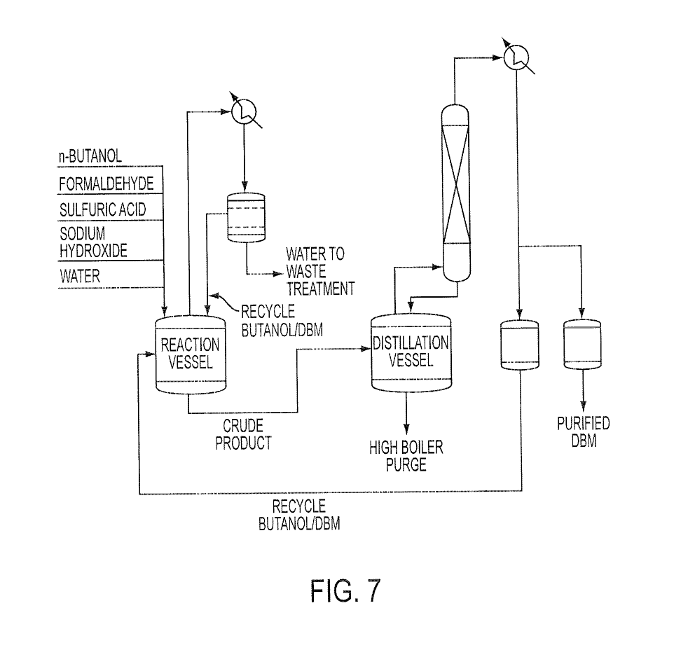

Generally speaking, manufacturing of the process of FIG. 2 can be easily conducted in either batch or continuous production. FIG. 6 shows an embodiment of a process flow diagram for the continuous production of dibutoxymethane based on the method discussed above. FIG. 7 shows an embodiment of a process flow diagram for the batch production of dibutoxymethane based on the methods discussed above.

Compared to the processes of the prior art, removal of the distilling agent is not a problem in the present process. Thus, advantages of the present process are that it requires fewer steps, such that manufacturing is easier and economically more feasible. Specifically, the advantage of this process over the prior art is that, unlike the prior art, an inert distilling agent is not required. The process of the present invention rather takes advantage of the heterogeneous butanol/water azeotrope. Due to the heterogeneous nature of the azeotrope, the butanol can then easily be recycled for subsequent production. This recycle allows for increased yields. Further, the lack of an inert distilling agent negates the need for a separation step from the product. Depending on the purity requirement of the product, it can be packaged without overhead distillation. Lastly, the process has flexible manufacturing options. In other words, the process has been designed so that manufacturing can be conducted easily in either batch or continuous equipment.

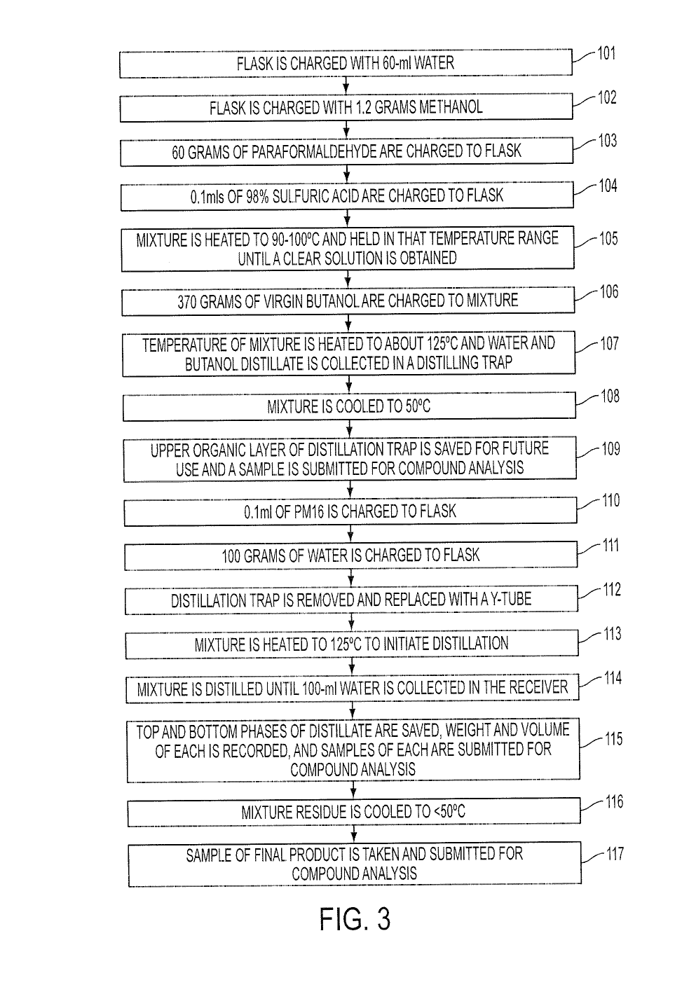

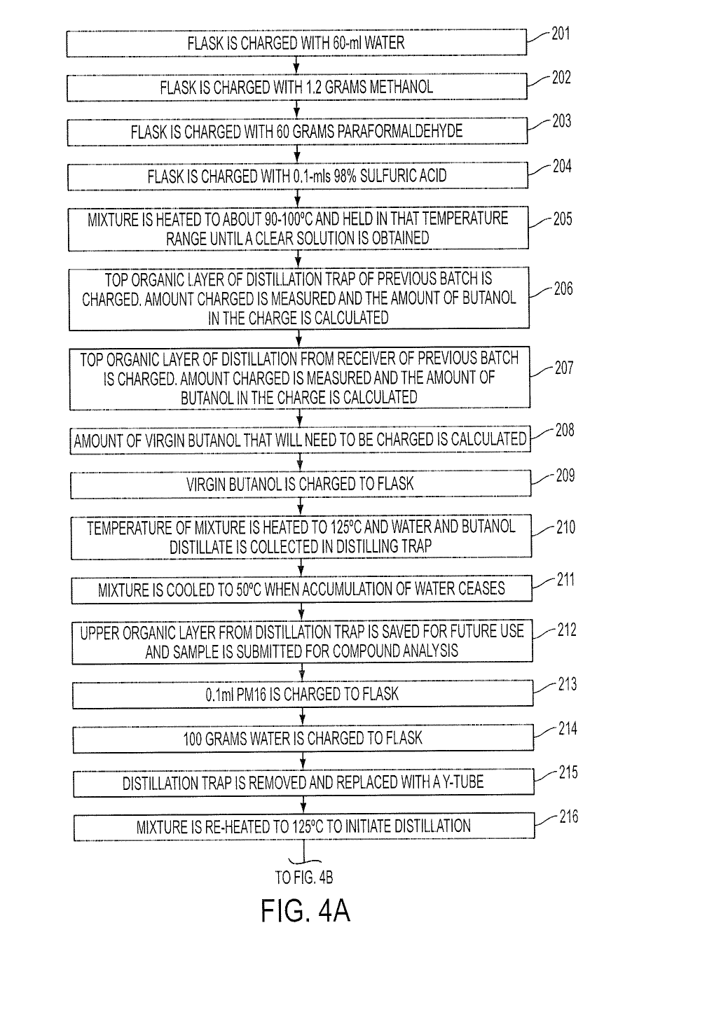

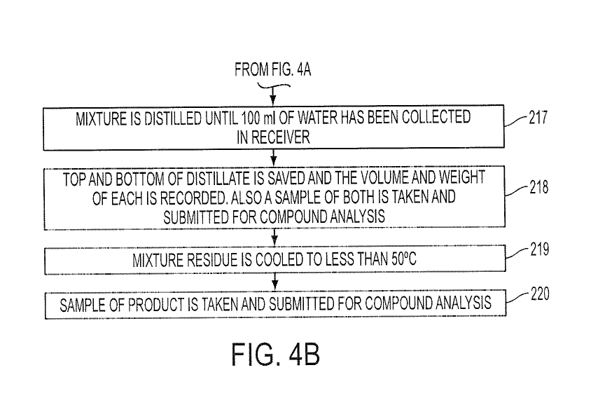

The following examples provide for embodiments of the processes disclosed here-in. The example depicted in FIG. 3 is an exemplary process without a distillate butanol residue recycle, it only utilizes virgin butanol. The example depicted in FIG. 4 is an exemplary process which utilizes a distillate butanol residue recycle in addition to virgin butanol. These processes are generally bench procedures and therefore are exemplary of what may be performed in production. It would be understood by one of ordinary skill in the art that these examples can be adapted to standard commercial operating processes. Further, for the purpose of this disclosure, it is noted that distillation and volume conditions discussed in this embodiment are not determinative, and any functional distillation or volume conditions known to those of skill in the art are contemplated in the processes of this disclosure. Moreover, it is inherent that any specifically identified flask, distillation column or other equipment is not determinative. Any piece of equipment known to those of skill in the art that can properly and effectively function in the given step of the disclosed processes is also contemplated.

### Example 1

To begin, in step (101), a flask is charged with 60-ml water. In the embodiment of the process depicted in FIG. 3, the flask is a 1-L flask. Further, in the embodiment of the process depicted in FIG. 3, deionized (“DI”) water is utilized, however it is important to emphasized that any water known to those of skill in the art is contemplated in this step.

Then, in step (102), the flask is charged with about 1.2 grams of methanol.

Next, about 60 grams of paraformaldehyde are charged to the flask in step (103) to create a 30-50% formaldehyde mixture.

Then, in step (104), 0.1-mls of 98% sulfuric acid are charged to the flask.

After the sulfuric acid is added, in step (105), the mixture is heated to about 90-100° C. and held in that temperature range until a clear solution is obtained.

Once the clear solution is obtained, in step (106), 370 grams of virgin butanol are charged to the mixture.

Then, in step (107), the temperature of the mixture is heated to about 125° C., and the resultant water and butanol distillate is collected in a distilling trap. Although the use of any distilling trap known to those of skill in the art is contemplated, in one embodiment, a Dean-Stark trap will be used.

In step (108), when the accumulation of the water ceases, the mixture will be cooled a temperature of about 50° C.

Then, in step (109), the upper organic layer from the distillation trap will be recycled to subsequent batches and a sample of the upper organic layer will be submitted for a compound analysis. While any compound analysis process known to those of skill in the art is contemplated in this step, in one embodiment the sample will be submitted for gas-liquid chromatography analysis.

Next, in step (110), 0.1-ml of 50% caustic will be charged to the flask.

Then, in step (111), 100 grams of water will be charged to the flask.

In the next step (112), the distillation trap will be removed from the reaction flask and replaced with a y-tube.

After the replacement is complete, the mixture will be re-heated to 125° C. to initiate distillation in step (113).

The mixture will continuously be distilled until about 100-ml of water has been collected in the receiver in step (114). Generally, it should be noted, that a two-phase distillate of about 250-ml total should be expected.

Then, in step (115), the top and bottom phases of the distillate will be saved. Also, in an embodiment of this step (115), the volume and weight of each of the top and bottom phases of the distillate will be recorded. Further, in an embodiment of this step (115), a sample of both the top and bottom distillate layers will be obtained and submitted for gas-liquid chromatography analysis. Lastly, it is contemplated in an embodiment that this step (115) will also consist of saving the upper organic phase for to be recycled in subsequent batches.

Next, in step (116), the mixture residue is cooled to about less than 50° C. and transferred to a storage bottle.

Then, in a final step (117), a sample of the product is taken and submitted for gas-liquid chromatography analysis.

While the expectant yield of the exemplary process depicted in FIG. 3 varies, in one embodiment it is expected to be about 320 grams.

### Example 2

To begin, in step (201), a flask is charged with 60-ml water. In the embodiment of the process depicted in FIG. 4, the flask is a 1-L flask. Further, in the embodiment of the process depicted in FIG. 4, deionized (“DI”) water is utilized, however it is important to emphasize that any water known to those of skill in the art is contemplated in this step.

Then, in step (202), the flask is charged with about 1.2 grams of methanol.

Next, about 60 grams of paraformaldehyde are charged to the flask in step (203) to create a mixture of 30-50% formaldehyde.

Then, in step (204), 0.1-mls of 98% sulfuric acid are charged to the flask.

After the sulfuric acid is added, in step (205), the mixture is heated to about 90-100° C. and held in that temperature range until a clear solution is obtained.

Once the clear solution is obtained, in step (206), the top, organic layer of the distillation trap distillate from the previous batch is charged. Also in this step (206), the amount charged is measured and the amount of butanol in the charge is calculated based on compound analysis, such as gas-liquid chromatography analysis.

Next, in step (207), the top organic layer from the receiver distillate from the previous batch is charged. Also in this step (207) the amount charged is measured and the amount of butanol in the charge is calculated based on compound analysis, such as gas-liquid chromatography analysis.

Then, in step (208), the amount of virgin butanol that will need to be charged to the mixture is calculated. Generally, this amount will be calculated by subtracting the combined net butanol charges from steps (206) and (207) from 370 grams.

After calculating the amount, the amount of virgin butanol calculated is charged to the flask in step (209).

Then, in step (210), the temperature of the mixture is heated to about 125° C., and the resultant water and butanol distillate is collected in a distilling trap. Although the use of any distilling trap known to those of skill in the art is contemplated, in one embodiment, a Dean-Stark trap will be used.

In step (211), when the accumulation of the water ceases, the mixture will be cooled a temperature of about 50° C.

Then, in step (212), the upper organic layer from the distillation trap will be recycled to subsequent batches and a sample of the upper organic layer will be submitted for a compound analysis. While any compound analysis process known to those of skill in the art is contemplated in this step, in one embodiment the sample will be submitted for gas-liquid chromatography analysis.

Next, in step (213), 0.1-ml of 50% caustic will be charged to the flask.

Then, in step (214), 100 grams of water will be charged to the flask.

In the next step (215), the distillation trap will be removed from the reaction flask and replaced with a y-tube.

After the replacement is complete, the mixture will be re-heated to 125° C. to initiate distillation in step (216).

The mixture will continuously be distilled until about 100-ml of water has been collected in the receiver in step (217). Generally, it should be noted, that a two-phase distillate of about 250-ml total should be expected.

Then, in step (218), the top and bottom phases of the distillate will be saved. Also, in an embodiment of this step (218), the volume and weight of each of the top and bottom phases of the distillate will be recorded. Further, in an embodiment of this step (218), a sample of both the top and bottom distillate layers will be obtained and submitted for gas-liquid chromatography analysis. Lastly, it is contemplated in an embodiment that this step (218) will also consist of saving the upper organic phase to be recycled with subsequent batches.

Next, in step (219), the mixture residue is cooled to about less than 50° C. and transferred to a storage bottle.

Then, in a final step (220), a sample of the product is taken and submitted for gas-liquid chromatography analysis.

While the expectant yield of the exemplary process depicted in FIG. 4 varies, in one embodiment it is expected to be about 320 grams.

While the invention has been disclosed in connection with certain preferred embodiments, this should not be taken as a limitation to all of the provided details. Modifications and variations of the described embodiments may be made without departing from the spirit and scope of the invention, and other embodiments should be understood to be encompassed in the present disclosure as would be understood by those of ordinary skill in the art.

1. A method for preparation of dibutoxymethane from 30-50% formaldehyde, the method comprising:
providing a 30-50% formaldehyde solution and butanol;
reacting said 30-50% formaldehyde solution and said butanol in a condensation reaction without the use of a co-solvent for the removal of water.

2. The method for the preparation of dibutoxymethane of claim 1, wherein said condensation reaction is performed with a recycled butanol discharge.

3. A dibutoxymethane formed by the process of:
providing a 30-50% formaldehyde and a butanol;
reacting said 30-50% formaldehyde and said butanol in a condensation reaction without the use of a co-solvent for the removal of water; and
segregating said dibutoxymethane.

4. The method for the preparation of dibutoxymethane of claim 3, wherein said condensation reaction is performed with a recycled butanol discharge.

5. A method for the production of dibutoxymethane, without a recycled butanol discharge, the method comprising the steps of:
charging water, methanol, paraformaldehyde, and a condensation reaction catalyst together to create a mixture;
heating said mixture to a temperature at which a clear solution is obtained;
charging virgin butanol to said mixture;
heating said mixture;
collecting water and butanol distillate in a distillation trap;
cooling said mixture when the accumulation of said water distillate ceases;
returning said butanol to said mixture;
charging a neutralization agent to said mixture;
charging water to said mixture;
re-heating said mixture to initiate distillation;
continuing said distillation until a predetermined amount of water has been collected;
separating a top and a bottom phase of a distillate; and
cooling a mixture residue.

6. The method of claim 5, wherein 60-ml of deionized water is charged in said step of charging.

7. The method of claim 5, wherein 1.2 grams of methanol are charged in said step of charging.

8. The method of claim 5, wherein 60 grams of paraformaldehyde are charged in said step of charging.

9. The method of claim 5, wherein 0.1 mls of 98% sulfuric acid are charged as said condensation reaction catalyst in said step of charging.

10. The method of claim 5, wherein said mixture is heated to a temperature of 90-100° C. in said step of heating said mixture to a temperature at which a clear solution is obtained.

11. The method of claim 5, wherein 370 grams of virgin butanol are charged to said mixture in said step of charging.

12. The method of claim 5, wherein said mixture is heated to a temperature of 125° C. in said step of heating said mixture.

13. The method of claim 5, wherein said mixture is cooled to 50° C. in said step of cooling said mixture when the accumulation of said water distillate ceases.

14. The method of claim 5, wherein 0.1-ml of 50% caustic is charged to said mixture in said step of charging a neutralization agent to said mixture.

15. The method of claim 5, wherein 110 grams of water are charged to said mixture in said step of charging water to said mixture.

16. The method of claim 5, wherein said mixture is re-heated to about 125° C. in said step of re-heating the mixture to initiate distillation.

17. The method of claim 5, wherein said distillation of said mixture continues until about 100 ml of water has been collected in said step of continuing distillation until water has been collected in a receiver.

18. The method of claim 5, wherein said mixture residue is cooled to less than 50° C. in said step of cooling a mixture residue.

19. A method for the production of dibutoxymethane, with a recycled butanol discharge, the method comprising the steps of:
charging water, methanol, paraformaldehyde, and a condensation reaction catalyst together to create a mixture;
heating said mixture to a temperature at which a clear solution is obtained;
charging a top layer of a distillate trap from a previous batch and calculating an amount of butanol therein;
charging a top layer from a receiver distillate from a previous batch and calculating an amount of butanol therein;
calculating an amount of virgin butanol to be charged;
charging the amount of said virgin butanol calculated in said step of calculating an amount of virgin butanol to be charged;
heating said mixture;
collecting water and butanol distillate in a distillation trap;
cooling said mixture when the accumulation of said water distillate ceases;
returning said butanol to said mixture;
charging a neutralization agent to said mixture;
charging water to said mixture;
re-heating said mixture to initiate distillation;
continuing said distillation until a predetermined amount water has been collected;
separating a top and a bottom phase of a distillate; and
cooling a mixture residue.
